(19) United States
(12) Patent Application Publication
(10) Pub. No.: US 2024/0316328 A1
(43) Pub. Date: Sep. 26, 2024

(54) Integrated Catheter Locking Mechanism for Port

(71) Applicant: Bard Peripheral Vascular, Inc., Franklin Lakes, NJ (US)

(72) Inventors: Christian Andersen, Queen Creek, AZ (US); Diana Fiumefreddo, Salt Lake City, UT (US); Ian N. Thomas, West Bountiful, UT (US); Jessica Hoye, Phoenix, AZ (US); Bryon Ray Densley, Rochester, MN (US)

(21) Appl. No.: 18/575,778

(22) PCT Filed: Jul. 7, 2021
(86) PCT No.: PCT/US21/40729

§ 371 Date: Dec. 29, 2023

(51) Int. Cl. A61M 39/02

(52) U.S. Cl. CPC A61M 39/0208

(57) ABSTRACT

Embodiments disclosed herein are directed to an integrated cathlock mechanism configured to engage an outer surface of a catheter to secure the catheter to a stem of a port, or similar medical device. The engagement structure can include a plurality of fins or an O-ring. The cathlock mechanism can include an engagement structure disposed annularly about the stem and configured to transition between an unlocked and locked configuration. The cathlock mechanism can be biased towards a locked configuration. A tab can selectively engage the cathlock mechanism and can maintain the cathlock mechanism in an unlocked configuration. Urging the catheter into the cathlock mechanism and onto the stem can engage the stem in an interference fit. A user can then remove the tab to transition the cathlock mechanism to the lock configuration.

### SUMMARY

Briefly summarized, embodiments disclosed herein are directed to an integrated catheter locking (“cathlock”) mechanism configured for coupling a proximal end of a catheter to a stem of a port, or similar medical device.

Proximally trimmable catheters allow for post-placement sizing of the catheter. When placing a catheter and port assembly, the position of the distal tip of the catheter can be important for the efficacy of the treatment. For example, when placing a catheter within the superior vena cava, if the distal tip of the catheter falls short of the target area, the efficacy of the medicament is reduced. If the distal tip is advanced too far, the distal tip can cause arrhythmia. The distance between the distal tip of the catheter and the port can vary since the distances between the target location, insertion site to the vasculature, and the location of the port can vary between patients and procedures. Estimating the catheter length before placement can lead to errors that result in misplacement of the distal tip.

Proximally trimmable catheters allow for placement of the catheter distal tip at the target location before trimming a proximal portion of the catheter to the correct length. The clinician can then attach the catheter to a subcutaneous port, or similar access device. However, securing the catheter to the port can be challenging. The connection must be leak-proof, especially under high-pressure infusion. Further, manipulating the catheter and port within the confined, wetted environment of a subcutaneous access site can lead to slippage, undue trauma to the access site, or misplacement of the catheter distal tip.

Disclosed herein is a locking mechanism for coupling a catheter to a port including, a stem extending along a longitudinal axis, a distal end of the stem configured to engage a lumen of the catheter, a proximal end of the stem coupled to the port, a cathlock having an engagement structure configured to engage the catheter and transitionable between an open configuration and a closed configuration and biased to the closed configuration, and a tab configured to engage the cathlock to maintain the engagement structure in an open configuration, the tab selectively removable from the cathlock to transition the engagement structure from the open configuration to the closed configuration.

In some embodiments, the cathlock is integrally formed with the port. In some embodiments, the cathlock includes a cowl extending from a body of the port and defining a recess, the recess extending longitudinally from a distal tip of the cowl and including the stem disposed therein. In some embodiments, the engagement structure includes one or more fins extending radially inward and elastically deformable to a radial outward open configuration. In some embodiments, the one or more fins extend at an angle relative to the longitudinal axis and either extend perpendicular to the longitudinal axis, or extend proximally toward the port.

In some embodiments, a rim of the one or more fins in the closed configuration defines a diameter that is equal to or less than an outer diameter of the stem. In some embodiments, a rim of the one or more fins in the closed configuration defines a diameter that is larger than an outer diameter of the stem and less than an outer diameter of the catheter. In some embodiments, the one or more fins includes a first series of fins disposed at a first longitudinal position within the cathlock and a second series of fins disposed at a second longitudinal position within the cathlock, different from the first longitudinal position.

In some embodiments, a first fin of the first series of fins is aligned with a first fin of the second series of fins along the longitudinal axis. In some embodiments, a first fin of the first series of fins is offset from a first fin of the second series of fins about the longitudinal axis of the stem. In some embodiments, a fin of the one or more fins extends through an arc distance of 360° about the axis of the stem. In some embodiments, a fin of the one or more fins extends through an arc distance of less than 360° about the axis of the stem. In some embodiments, the engagement structure includes an O-ring extending annularly about the axis of the stem, and elastically deformable to a radial outward open configuration.

In some embodiments, the O-ring in the closed configuration defines an inner diameter that is less than an outer diameter of the catheter. In some embodiments, the tab is configured to reengage the cathlock, after it has been removed therefrom, to transition the cathlock from the closed position to the open position. In some embodiments, the tab includes a frangible bridge coupling the tab to the cathlock, and configured to separate when the tab is selectively removed from the cathlock. In some embodiments, the tab includes a shaft extending longitudinally and defining a tab lumen, an inner diameter of the tab lumen being larger than an outer diameter of the catheter.

In some embodiments, the tab includes an elongate opening extending longitudinally and communicating between an outer surface thereof and the tab lumen, the elongate opening configured to allow ingress or egress of the catheter from the tab lumen. In some embodiments, the elongate opening defines a width that is less than an outer diameter of the catheter. In some embodiments, the elongate opening defines a width that is equal to or larger than an outer diameter of the catheter. In some embodiments, the tab further includes a handle extending from a distal end of the shaft, perpendicular to the longitudinal axis, and configured to facilitate grasping the tab.

Also disclosed is a method of coupling a catheter to a port including, urging the catheter onto a stem of the port, the catheter engaging the stem in an interference fit, removing a pull tab from a cathlock mechanism, the cathlock mechanism biased towards a locked configuration, the pull tab configured to engage the cathlock mechanism and maintain the cathlock mechanism in an unlocked configuration, and transitioning the cathlock mechanism to a locked configuration.

In some embodiments, the cathlock mechanism is integrally formed with the port. In some embodiments, the cathlock mechanism includes a cowl extending from a body of the port and defining a recess, the recess extending longitudinally from a distal tip of the cowl and including the stem disposed therein. In some embodiments, the cathlock mechanism includes one or more fins extending radially inward and elastically deformable to a radial outward unlocked configuration. In some embodiments, a rim of the one or more fins in the locked configuration defines a diameter that is equal to or less than an outer diameter of the catheter.

In some embodiments, the engagement structure includes an O-ring extending annularly about the axis of the stem, and elastically deformable to a radial outward unlocked configuration. In some embodiments, the O-ring in the locked configuration defines an inner diameter that is less than an outer diameter of the catheter. In some embodiments, the method further includes reengaging the tab with the cathlock to transition the cathlock from the locked position to the unlocked position. In some embodiments, the method further includes separating a frangible bridge coupling the tab to the cathlock before removing the tab from the cathlock mechanism.

In some embodiments, the method further includes urging the catheter through an elongate opening of the tab, the elongate opening communicating with a tab lumen. In some embodiments, the elongate opening defines a width that is less than an outer diameter of the catheter. In some embodiments, the tab further includes a handle extending perpendicular to the longitudinal axis, and configured to facilitate removing the tab from the cathlock mechanism.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a port including an integrated cathlock mechanism, in accordance with embodiments disclosed herein.

FIG. 1B shows a cross-section perspective view of a port including an integrated cathlock mechanism, in accordance with embodiments disclosed herein.

FIG. 2 shows a longitudinal cross-section view of a port including an integrated cathlock mechanism, in accordance with embodiments disclosed herein.

FIGS. 3A-3B shows an exemplary method of use of an integrated cathlock mechanism, in accordance with embodiments disclosed herein.

FIG. 3C shows an integrated cathlock mechanism including a pull tab, in accordance with embodiments disclosed herein.

FIG. 4A shows a lateral cross-sectional view of an integrated cathlock mechanism, in accordance with embodiments disclosed herein.

FIG. 4B shows a lateral cross-sectional view of an integrated cathlock mechanism, in accordance with embodiments disclosed herein.

FIG. 5A shows a perspective view of a port including an integrated cathlock mechanism, in accordance with embodiments disclosed herein.

FIG. 5B shows a plan view of the port of FIG. 5A, in accordance with embodiments disclosed herein.

FIG. 5C shows a distal end view of the port of FIG. 5A, in accordance with embodiments disclosed herein.

FIG. 6 shows a perspective view of a pull tab, in accordance with embodiments disclosed herein.

FIGS. 7A-7B show an exemplary method of use for an integrated cathlock mechanism, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

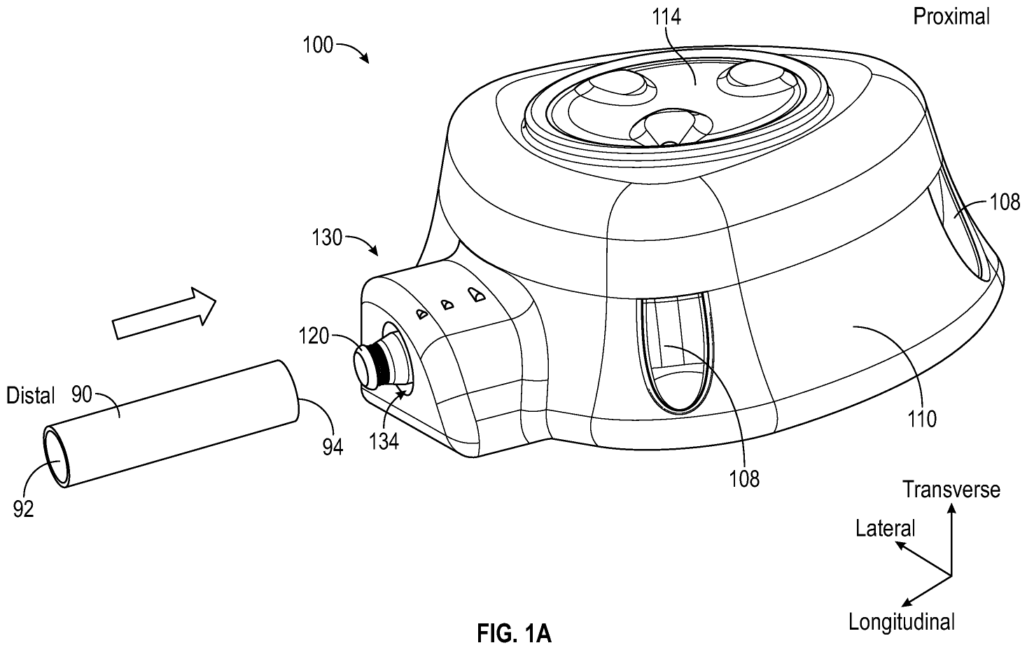

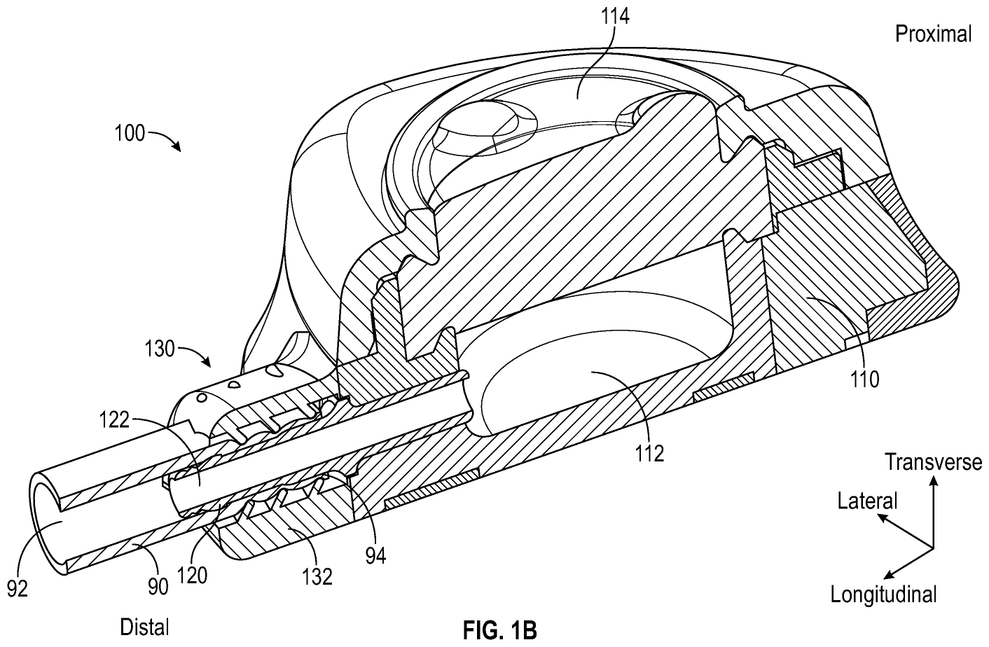

To assist in the description of embodiments described herein, as shown in FIGS. 1A-1B, a longitudinal axis extends substantially parallel to an axial length of the stem 120. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

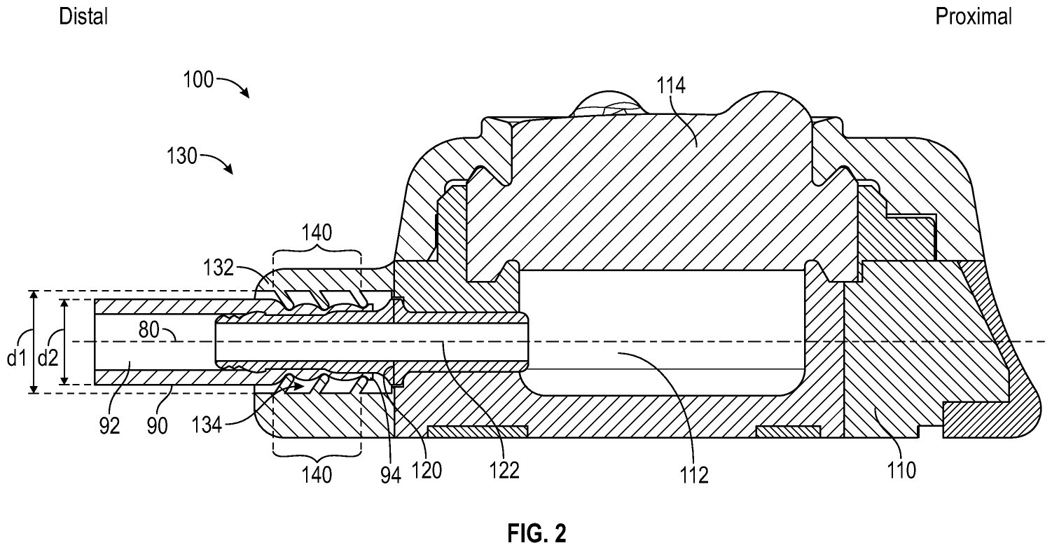

FIGS. 1A-2 show an embodiment of a port 100 including an integrated catheter locking (“cathlock”) mechanism 130 configured to secure a catheter 90 to a stem 120. FIG. 1A shows a perspective view of the port 100. FIGS. 1B-2 show longitudinal cross-section views of the port 100. The port 100 can generally include a port body 110 that defines a reservoir 112 and can include a needle penetrable septum 114 disposed thereover. The septum 114 can be configured to provide percutaneous access to the reservoir 112 by an access needle. The access needle can penetrate the skin surface and underlying tissues and can be urged through the needle penetrable septum 114 to access the reservoir 112 and provide fluid communication therewith.

The port 100 can further include a stem 120 defining a stem lumen 122 that is in fluid communication with the reservoir 112. In an embodiment, a proximal end 94 of the catheter 90 can be urged over the stem 120 to provide fluid communication between the reservoir 112 and a lumen 92 of the catheter 90. A distal tip of the catheter 90 can be disposed within a vasculature of a patient to provide fluid communication therewith. It will be appreciated that the subcutaneous port 100 is an exemplary medical device and that embodiments disclosed herein can be used with various medical devices that require a compliant tube to be fluidly coupled with a rigid stem to provide fluid communication therebetween.

In an embodiment, the port 100 can include an integrated cathlock mechanism 130. The cathlock mechanism 130 can include a cowl 132, extending distally from the port body 110 and extending annularly about the stem 120. In an embodiment, a distal tip of the cowl 132 can extend to a point that is proximal of the distal tip of the stem 120. In an embodiment, a distal tip of the cowl 132 can extend to a point that is distal of the distal tip of the stem 120. In an embodiment, a distal tip of the cowl 132 and a distal tip of the stem 120 can extend equidistant from the body 110.

In an embodiment, the cowl 132 can define a substantially cylindrical recess 134 extending longitudinally from the distal tip of the cowl 132 and can include the stem 120 disposed therein. In an embodiment, the recess 134 can define a substantially circular or elliptical cross-sectional shape. However, other cross-sectional shapes are also contemplated. In an embodiment, the recess 134 can define a diameter (d1) which can be larger than an outer diameter (d2) of the catheter 90.

In an embodiment, the cathlock mechanism 130 can further include an engagement structure 140 disposed within the recess 134 and configured to engage an outer surface of the catheter 90 and secure the catheter 90 to the stem 120. The engagement structure 140 can be configured to transition between an open, or unlocked, configuration and a closed, or locked configuration. In an embodiment, the engagement structure 140 can be biased to the closed, or locked configuration.

In an embodiment, the engagement structure 140 can include one or more fins 142 extending radially inward from an inner surface of the recess 134. In an embodiment, the one or more fins 142 can be formed of a plastic, polymer, elastomer, metal, alloy, composite, or the like. In an embodiment, the one or more fins 142 can extend at an angle, relative to a central longitudinal axis 80. In an embodiment, the one or more fins 142 can extend perpendicular to the central axis 80. In an embodiment, the one or more fins 142 can extend at an angle extending towards the port body 110, i.e. in a proximal direction. In an embodiment, the one or more fins 142 can be configured to elastically deform radially outward, from the closed position to the open position.

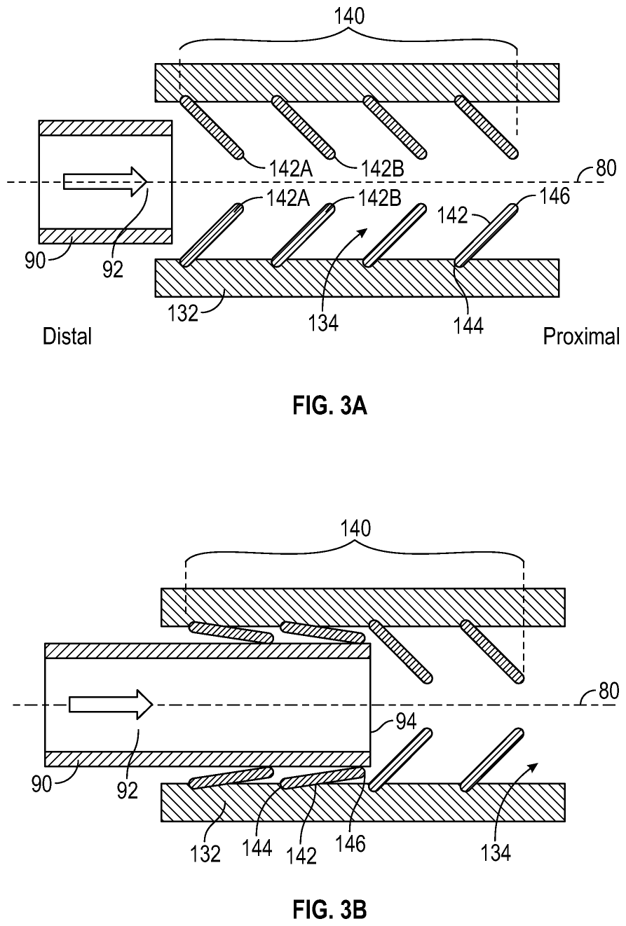

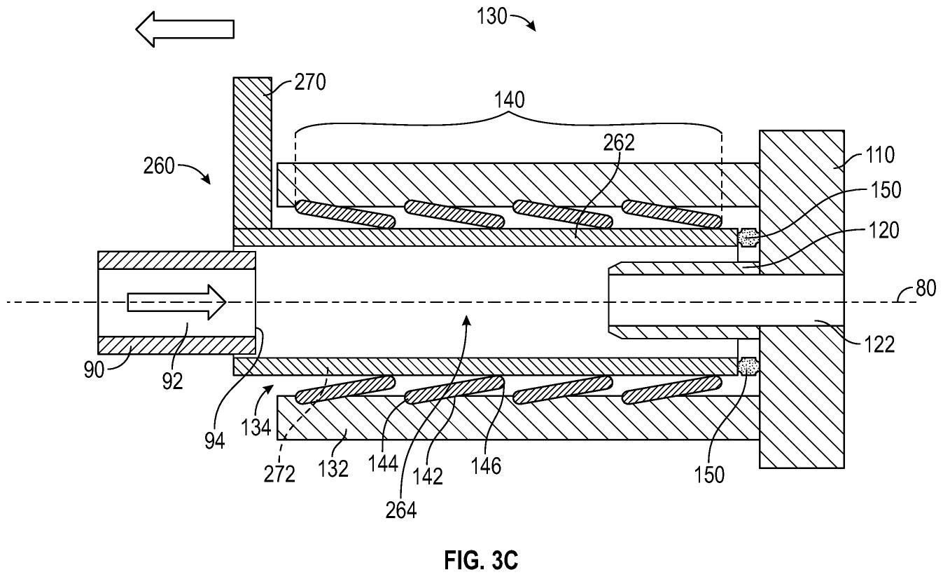

In an exemplary method of use, as shown in FIGS. 3A-3B, the cathlock mechanism 130 can be provided including one or more fins 142 biased towards the closed position (FIG. 3A). Each fin of the one or more fins 142 can be coupled to an inner wall of the recess 134 at a base 144 and can extend radially inward to a rim 146 disposed. To note, for clarity, FIGS. 3A-3D show the engagement structure 140 without the stem 120. However, as will be appreciated, the stem 120 can extend through the recess 134 to engage the catheter 90. Advantageously, the stem 120 can provide columnar support to the catheter 90 as the proximal end 94 of the catheter 90 is urged into the recess 134.

A user can urge a proximal end 94 of the catheter 90 into the recess 134 and past one or more fins 142. The catheter 90 can elastically deform the one or more fins 142, transitioning from the closed position to the open position (FIG. 3B). In an embodiment, each fin of the one or more fins 142 can be coupled to the inner wall of the recess 134 at a base 144 by a hinge or living hinge, configured to allow the one or more fins 142 to pivot about the base 144 and transition between the closed and open positions.

In an embodiment, a catheter 90 can be urged into the recess 134 of the cathlock mechanism 130 and the stem 120 can engage a lumen 92 of the catheter 90. The outer diameter of the stem 120 can be the same or slightly larger than the inner diameter of the lumen 92 of the catheter 90. The stem 120 can be formed of a resilient or rigid material. The catheter 90 can be formed of a compliant, or elastically deformable material. As such, the catheter 90 can elastically deform to fit over the stem 120 and engage the stem 120 in an interference fit.

The one or more fins 142, biased towards the closed configuration can engage an outer surface of the catheter 90 and further secure the catheter 90 thereto. In an embodiment, the one or more fins 142 can extend perpendicular to the central axis 80 and can compress the catheter 90 on to the stem while still allowing movement of the catheter 90 in both the proximal and distal directions, e.g. if the catheter 90 were to be removed from the port 100 and exchanged. In an embodiment, as shown in FIG. 3A, the one or more fins 142 can extend at an angle towards the port body 110, i.e. in a proximal direction. Advantageously, the fins 142 can allow movement of the catheter 90 in a proximal direction but can mitigate movement of the catheter 90 is a distal direction, mitigating accidental disengagement of the catheter 90 from the port 100.

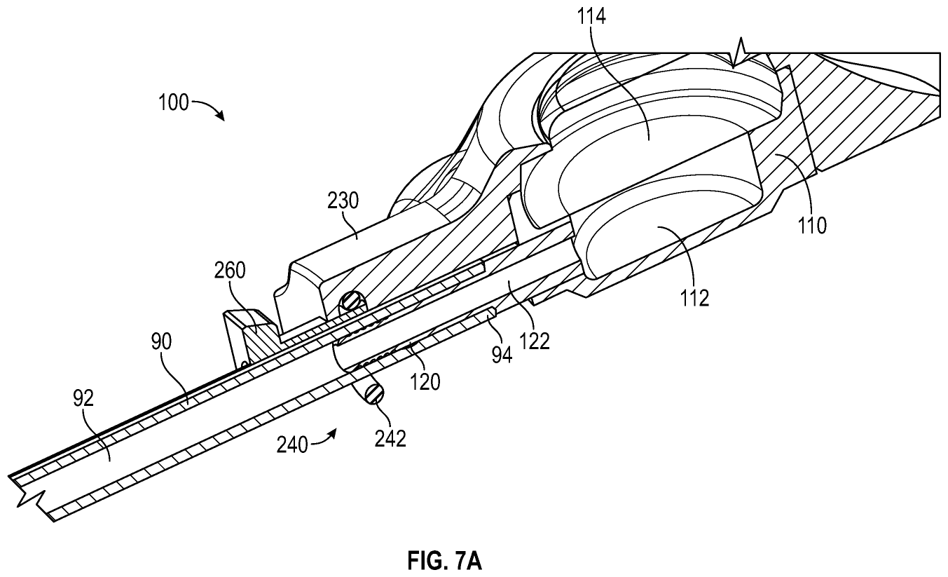

In an embodiment, as shown in FIGS. 3C, 6, the cathlock mechanism can further include a pull tab (“tab”) 260 configured to maintain the engagement structure in an open, or unlocked, configuration.

The catheter 90 can be advanced into a lumen 264 of the tab 260 and engage the stem 120 in an interference fit, as described herein. The tab 260 can then be removed from the recess 134 by a user grasping the handle 270 and urging the tab distally. In an embodiment, the tab 260 can be releasably coupled to the cathlock mechanism 130 by a frangible bridge 150. The frangible bridge 150 can prevent premature disengagement of the tab 260 from the cathlock mechanism 130. Urging the tab 260 distally from the recess 134 can cause the frangible bridge 150 to separate from the cathlock mechanism 130 releasing the tab 260.

In an embodiment, the tab 260 can be re-engaged with the cathlock mechanism 130 to transition the engagement structure 140 from the locked position to the unlocked position, either before or after the catheter 90 is engaged with the stem 120. For example, the port 100 including the cathlock mechanism 130 can be provided with the engagement structure 140 in the normally-closed position (FIG. 3A). The user can insert the tab 260 into the recess 134 to transition the engagement structure 140 from the closed to the open position. The catheter 90 can then be inserted into the lumen 264 of the tab 260 and engaged with the stem 120. The tab 260 can be removed to transition the engagement structure 140 from the open position to the closed position. In an embodiment, the tab 260 can include an elongate opening 272 configured to allow the tab 260 to selectively engage or disengage the catheter 90. In an embodiment, the tab 260 can then be reinserted into the recess 134 and transition the engagement structure from the closed position to the open position. The catheter 90 can then be urged distally to disengage the stem 120 and disengage the cathlock mechanism 130.

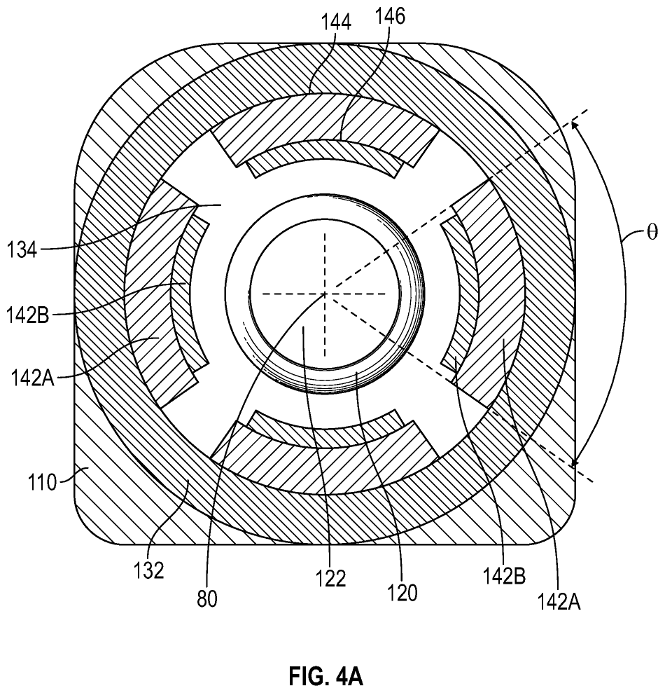

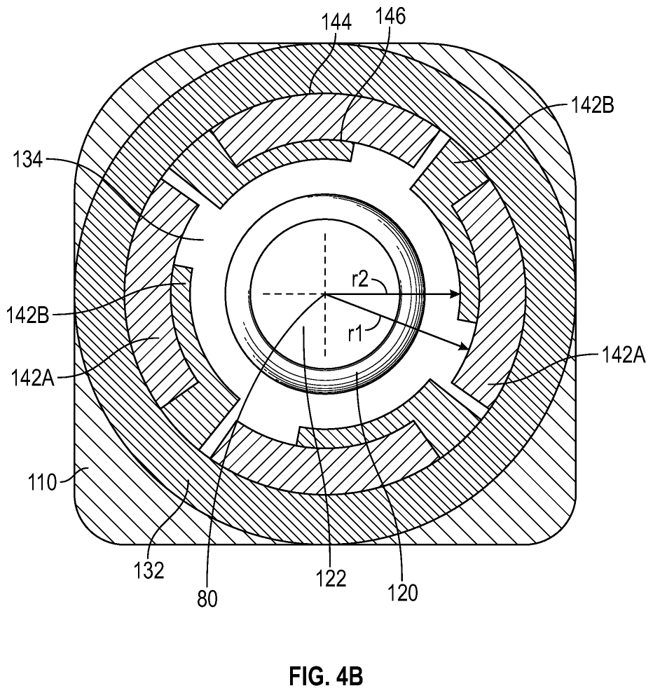

In an embodiment, as shown in FIGS. 4A-4B, each fin of the one or more fins 142 can extend annularly about the central axis 80 through an arc distance (Θ). In an embodiment, the arc distance (Θ) can be between 5° and 360°. In an embodiment, a first series of fins 142A can be disposed at a first longitudinal position within the recess 134 and can include one or more fins disposed radially about the central axis 80. Each fin of the first series of fins 142A can extend through an arc distance (Θ) of 360° or less. In an embodiment, a second series of fins 142B can be disposed at a second longitudinal position within the recess 134, different from the first longitudinal position, and can include one or more fins disposed radially about the central axis 80. Each fin of the second series of fins 142A can extend through an arc distance (Θ) of 360° or less.

In an embodiment, as shown in FIG. 4A, a first fin of the first series of fins 142A can be aligned with a first fin of the second series of fins 142B along a longitudinal axis. i.e. disposed at the same radial position about the central axis 80. In an embodiment, as shown in FIG. 4B, a first fin of the first series of fins 142A can be radially offset from a first fin of the second series of fins 142B along a longitudinal axis. In an embodiment, an inner rim 146 of a fin of the first series of fins 142A can extend radially inward by a first radius (r1), an inner rim 146 of a fin of the second series of fins 142B can extend radially inward by a second radius (r2). The first radius (r1) can be greater than, less than, or equal to the second radius (r2). In an embodiment, an inner rim 146 of the one or more fins 142 can define a diameter that is equal to or larger than an outer diameter of the stem 120. In an embodiment, an inner rim 146 of the one or more fins 142 can define a diameter that is equal to or less than an outer diameter (d2) of the catheter 90.

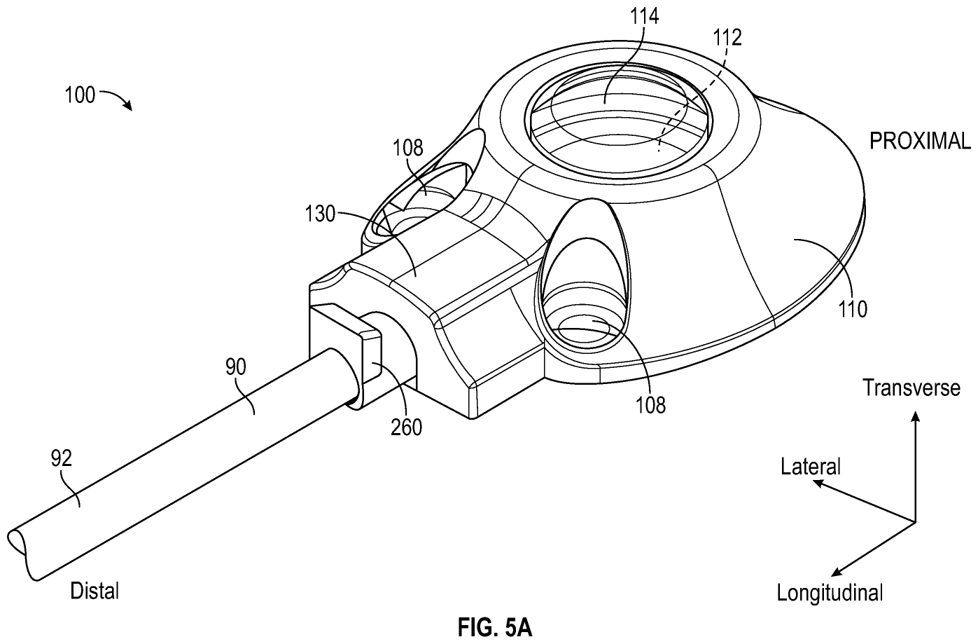

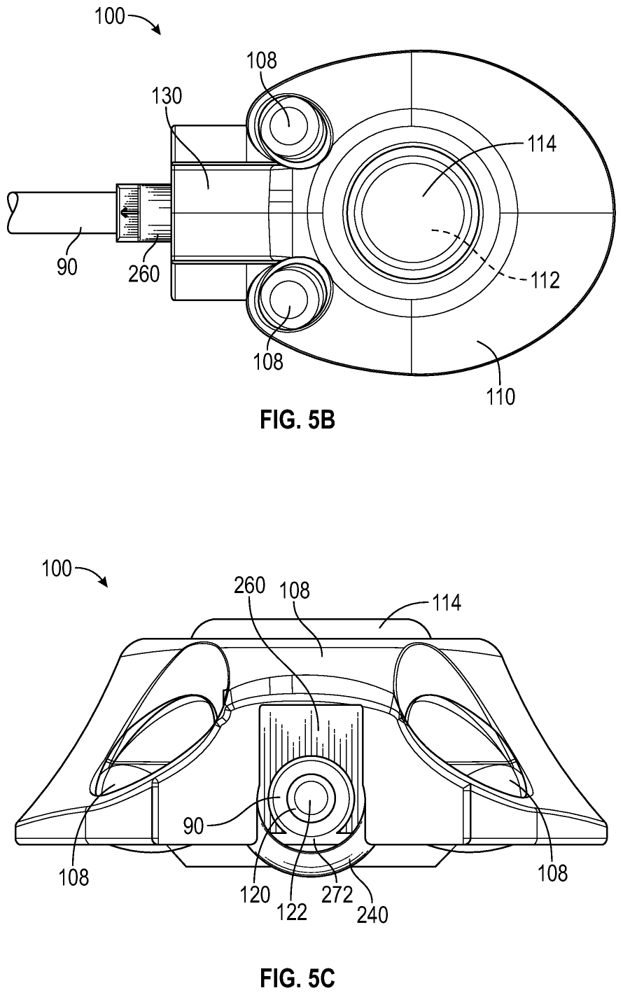

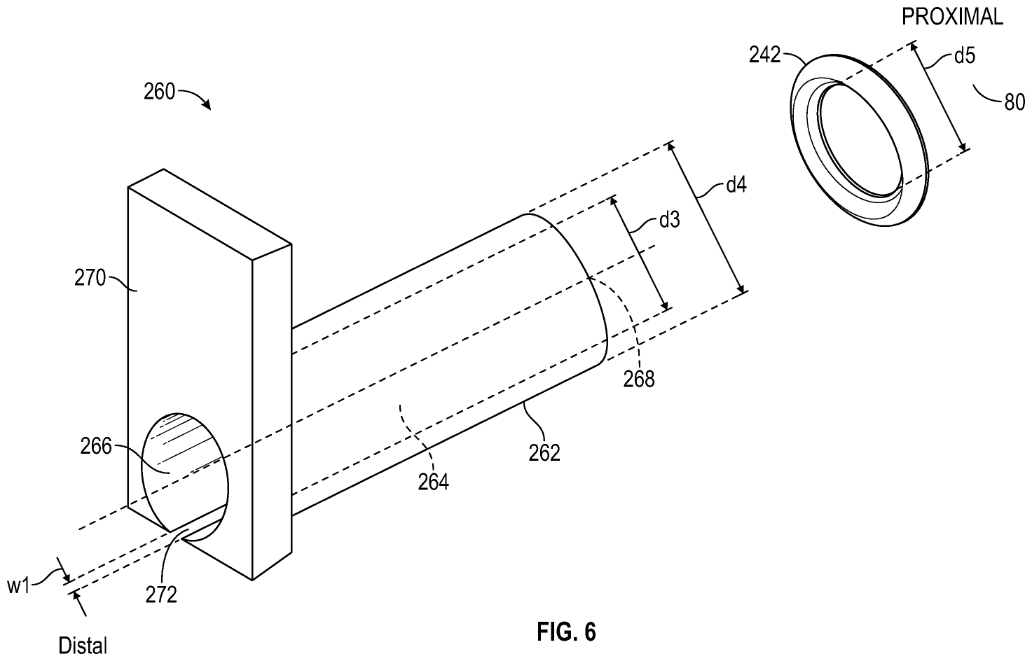

FIGS. 5A-7B show an embodiment of a port 100 including an integrated cathlock mechanism 130 having an O-ring engagement structure 240. The port 100 can include a stem 120 defining a stem lumen 122 that is in fluid communication with a reservoir 112. The port 100 can further include an integrated cathlock mechanism 130 extending therefrom and including a cowl 132 defining a recess 134, and encircles the stem 120, as described herein.

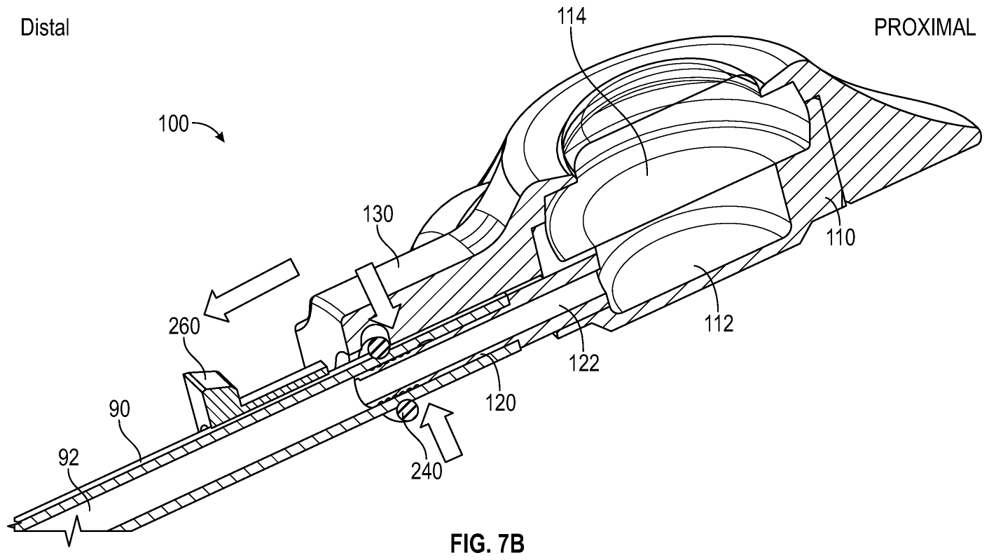

In an embodiment, the cathlock mechanism 130 can further include an O-ring engagement structure 240 disposed within the recess 134 and extending annularly about the central axis 80. The O-ring engagement structure 240 can include one or more O-rings 242 formed of an elastically deformable material and transitionable between an expanded, open or unlocked configuration (FIG. 7A), and a retracted, closed or locked configuration (FIG. 7B). In an embodiment, the O-ring 242 can be biased towards the locked configuration.

In an embodiment, the cathlock mechanism 130 can further include a tab 260, as described herein. As shown in FIG. 6, the tab 260 can include a shaft 262 defining a lumen 264 extending longitudinally between a distal opening 266 and a proximal opening 268. The tab 260, or portions thereof, can be formed of a resilient or rigid material such as a plastic, polymer, metal, alloy, composite, or the like. In an embodiment, an inner diameter (d3) of the tab lumen 264 can be larger than an outer diameter (d2) of the catheter 90. An outer diameter (d4) of the tab shaft 262 can be less than an inner diameter (d1) of the recess 134.

The tab 260 can further include a handle 270 coupled to a distal end of the shaft 262, and extending perpendicular to the longitudinal axis. The handle 270 can be configured to allow a user to manipulate the tab 260. In an embodiment, the handle 270 can include a finger loop, protrusions, abutments, ridges, or the like, or can include one or more materials with different friction co-efficient (e.g. silicone rubber) to facilitate grasping the handle 270, especially within the confined wetted environment of a subcutaneous placement. In an embodiment, the shaft 262 can be configured to extend into the recess 134 and extend through the O-ring 242 to maintain the O-ring 242 in an open configuration. In an embodiment, the outer diameter (d4) of the shaft can be larger than an inner diameter (d5) of the O-ring 242 in the closed configuration.

In an embodiment, the tab 260 can further include an elongate opening 272 extending longitudinally along a bottom surface of the tab 260 between the distal opening 266 and the proximal opening 268, and communicating with the tab lumen 264. In an embodiment, the elongate opening 272 can define a lateral width (w1) that is equal to or larger than an outer diameter (d2) of the catheter 90. As such, the elongate opening 272 can allow ingress or egress of the catheter 90 to/from the tab lumen 264. In an embodiment, the lateral width (w) of the elongate opening 272 can be smaller than the outer diameter (d2) of the catheter 90. As such, the catheter 90 can be urged through the elongate opening 272, and the catheter 90 can elastically deform to pass therethrough. Advantageously, the lateral width (w1) being less than the outer diameter (d2) can prevent unintentional disengagement of the tab 260 from the catheter 90. In an embodiment, the tab lumen 264 having the elongate opening 272 disposed therein, extends annularly about the central axis 80 through 180° or more.

In an exemplary method of use, a port 100 can be provided including a cathlock mechanism 230, as described herein. In an embodiment, cathlock mechanism 130 can further include a tab 260 disposed within the recess 134 and configured to maintain the engagement structure 240 in an open configuration. For example, as shown in FIG. 7A, the tab shaft 262 can extend longitudinally into the recess 134 and elastically deform the O-ring 242 radially outward to the unlocked or open configuration, i.e. to an outer diameter (d4) of the tab shaft 262.

In an embodiment, the tab 260 can be “pre-loaded” within the recess 134, i.e. assembled during manufacture with the engagement structure 240 is maintained in the open configuration during transport and storage. Advantageously, this can simplify the coupling process during use since the cathlock mechanism 130 is ready for use, the user simply has to engage the catheter with the stem 120 and remove the tab 260, as described in more detail herein.

In an embodiment, the tab 260 can be assembled and placed within the recess 134 by the clinician prior to engagement with the catheter 90. Worded differently, the engagement structure 240 is maintained in the closed configuration during transport and storage and transitioned to the open position just prior to use. Advantageously, this can mitigate material fatigue, or “creep,” during storage since the engagement structure is not maintained in a stressed state for a prolonged period of time.

With the tab 260 maintaining the engagement structure 240 in the open configuration, the user can slide the catheter 90 longitudinally through the distal opening 266 and into the tab lumen 264 until the proximal end 94 engages the stem 120. With the catheter 90 engaged with the stem 120, the user can remove the tab 260 from the recess 134 by grasping the handle 270 and urging the tab 260 distally. As the tab 260 is removed, the engagement structure 240 can transition from the open position to the closed position. For example, the O-ring 242 that was elastically deformed to the extended configuration can return to the retracted or closed configuration (FIG. 7B). In the closed position, the O-ring 242 can tighten annularly about the outer surface of the catheter 90, securing the catheter 90 onto the stem 120.

The tab 260 can then be slid distally out of the recess 134 and the catheter 90 can be urged through the elongate opening 272 to disengage the port 100/catheter 90 assembly. In an embodiment, the tab 260 can be reengaged with the catheter 90 by urging a portion of the catheter 90 through the elongate opening 272. The tab 260 can then be urged proximally along an outer surface of the catheter 90 to reengage the cathlock mechanism 130. A proximal end of the shaft 262 can include a chamfered edge to facilitate urging the shaft 262 longitudinally between the catheter 90 and the engagement structure 240 to transition the engagement structure 240 from the closed position to the open position. The catheter 90 can then be disengaged from the stem 120.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A locking mechanism for coupling a catheter to a port, comprising:
a stem extending along a longitudinal axis, a distal end of the stem configured to engage a lumen of the catheter, a proximal end of the stem coupled to the port;
a cathlock having an engagement structure configured to engage the catheter and transitionable between an open configuration and a closed configuration and biased to the closed configuration; and
a tab configured to engage the cathlock to maintain the engagement structure in an open configuration, the tab selectively removable from the cathlock to transition the engagement structure from the open configuration to the closed configuration.

2. The locking mechanism according to claim 1, wherein the cathlock is integrally formed with the port.

3. The locking mechanism according to claim 2, wherein the cathlock includes a cowl extending from a body of the port and defining a recess, the recess extending longitudinally from a distal tip of the cowl and including the stem disposed therein.

4. The locking mechanism according to claim 1, wherein the engagement structure includes one or more fins extending radially inward and elastically deformable to a radial outward open configuration.

5. The locking mechanism according to claim 4, wherein the one or more fins extend at an angle relative to the longitudinal axis and either extend perpendicular to the longitudinal axis, or extend proximally toward the port.

6. The locking mechanism according to claim 4, wherein a rim of the one or more fins in the closed configuration defines a diameter that is equal to or less than an outer diameter of the stem.

7. The locking mechanism according to claim 4, wherein a rim of the one or more fins in the closed configuration defines a diameter that is larger than an outer diameter of the stem and less than an outer diameter of the catheter.

8. The locking mechanism according to claim 4, wherein the one or more fins includes a first series of fins disposed at a first longitudinal position within the cathlock and a second series of fins disposed at a second longitudinal position within the cathlock, different from the first longitudinal position.

9. The locking mechanism according to claim 8, wherein a first fin of the first series of fins is aligned with a first fin of the second series of fins along the longitudinal axis.

10. The locking mechanism according to claim 8, wherein a first fin of the first series of fins is offset from a first fin of the second series of fins about the longitudinal axis of the stem.

11. The locking mechanism according to claim 4, wherein a fin of the one or more fins extends through an arc distance of 360° about the axis of the stem.

12. The locking mechanism according to claim 4, wherein a fin of the one or more fins extends through an arc distance of less than 360° about the axis of the stem.

13. The locking mechanism according to claim 1, wherein the engagement structure includes an O-ring extending annularly about the axis of the stem, and elastically deformable to a radial outward open configuration.

14. The locking mechanism according to claim 13, wherein the O-ring in the closed configuration defines an inner diameter that is less than an outer diameter of the catheter.

15. The locking mechanism according to claim 1, wherein the tab is configured to reengage the cathlock, after it has been removed therefrom, to transition the cathlock from the closed position to the open position.

16. The locking mechanism according to claim 1, wherein the tab includes a frangible bridge coupling the tab to the cathlock, and configured to separate when the tab is selectively removed from the cathlock.

17. The locking mechanism according to claim 1, wherein the tab includes a shaft extending longitudinally and defining a tab lumen, an inner diameter of the tab lumen being larger than an outer diameter of the catheter.

18. The locking mechanism according to claim 17, wherein the tab includes an elongate opening extending longitudinally and communicating between an outer surface thereof and the tab lumen, the elongate opening configured to allow ingress or egress of the catheter from the tab lumen.

19. The locking mechanism according to claim 18, wherein the elongate opening defines a width that is less than an outer diameter of the catheter.

20. The locking mechanism according to claim 18, wherein the elongate opening defines a width that is equal to or larger than an outer diameter of the catheter.

21. The locking mechanism according to claim 17, wherein the tab further includes a handle extending from a distal end of the shaft, perpendicular to the longitudinal axis, and configured to facilitate grasping the tab.

22-33. (canceled)
